(19) United States
(12) Patent Application Publication
(10) Pub. No.: US 2003/0083644 A1
(43) Pub. Date: May 1, 2003

(54) Sterilizable device for draining urine

(76) Inventor: Paolo Avaltroni, Mantova (IT)

Correspondence Address: William J. Sapone, Esq., at the offices of Coleman Sudol Sapone P.C., 714 Colorado Ave., Bridgeport, CT, 06605, US

(21) Appl. No.: 10/285,091

(22) Filed: Oct. 31, 2002

(30) Foreign Application Priority Data
Oct. 31, 2001 (IT) BO2001U 000091

(51) Int. Cl. A61F 5/44; A61M 27/00

(52) U.S. Cl. 604/544; 604/328

(57) ABSTRACT

A sterilizable device for draining urine includes a collecting bag sealed until the moment in which the catheter is to be used. The bag, in turn, includes an elongated neck-like portion and a collecting portion, joined to the elongated portion and designed for collection of urine drained by the catheter. The catheter is packed inside the collecting bag in the region corresponding to the elongated portion, and can be extracted therefrom when the device is to be used. The elongated portion has an opening at its distal end for allowing extraction of the catheter. The device includes also a segment of liquid-impermeable material removably fastened to the elongated portion at its distal end, so as to seal the opening.

### BACKGROUND OF THE INVENTION

The present invention relates to the manufacture of paramedical products and, in particular, of devices for draining urine.

In detail, the invention concerns a new disposable sterilizable device for draining urine, the device including a collection bag and a catheter.

### DESCRIPTION OF THE PRIOR ART

It is known that catheters, in particular disposable catheters, which are used to drain urine, are usually packed within flexible bags. The bags are then air-tightly sealed by heat-welding or high frequency-welding. The bags are so shaped as to form an elongated neck portion, designed to retain a catheter thereinside, joined to a larger portion, designed to collect the urine drained by means of the catheter. At the distal end of the neck portion, in the region corresponding to the tip portion of the catheter, there is a small slit, which forms a rupture-facilitated section.

Inside the collection portion, there is also a small container filled with sterile water. Also the small container is tightly closed. The container is usually formed from hard plastic material and features a precut line, which makes it easier to detach a part of the container. In this way, the liquid inside the container is released.

When the catheter is to be used, first the container inside the bag must be opened to let the water contained therein expand in the bag. As soon as the water contacts the catheter, a lubricating substance, previously applied to its outer surface, becomes active to facilitate insertion of the catheter in its operative seat.

Then, the distal end of the collecting bag neck portion is ruptured by tearing it crosswise, with the help of the above-mentioned slit. The catheter is then extracted and a final part, which is flared, is engaged by the opening and remains inside the elongated portion of the bag. The catheter is used and then disposed in accordance to the usual ways, which are commonly known.

A drawback often encountered when the above-mentioned package is used, derives from the form of the distal end of the bag, which is sometime difficult to open or defective. This can be usually caused by either faulty making of the slit or by lack of skill. More commonly, this problem occurs because of the partial physical inability, typical of the common user of this type of device, who is often affected by difficulties in coordination of movements. In such cases, the bag can result to be difficult to rupture or can rip along a wrong line, for instance longitudinally, so that the bag becomes unusable.

### SUMMARY OF THE INVENTION

It is an object of the present invention to provide a new sterilizable device for draining urine, which device overcomes all the drawbacks mentioned above.

Another object of the invention is to provide a device for draining urine, which involves low costs for production and, consequently, also for sale.

The above mentioned objects are achieved in accordance with the contents of the claims, by means of

### BRIEF DESCRIPTION OF THE DRAWINGS

The invention will now be described in more detail with reference to particular, non-limiting embodiments and with reference to the accompanying drawings, in which:

FIG. 1 shows schematically a lateral view of the device for draining urine manufactured in accordance with the present invention;

FIG. 2 shows an enlarged view of a collecting bag elongated portion distal end;

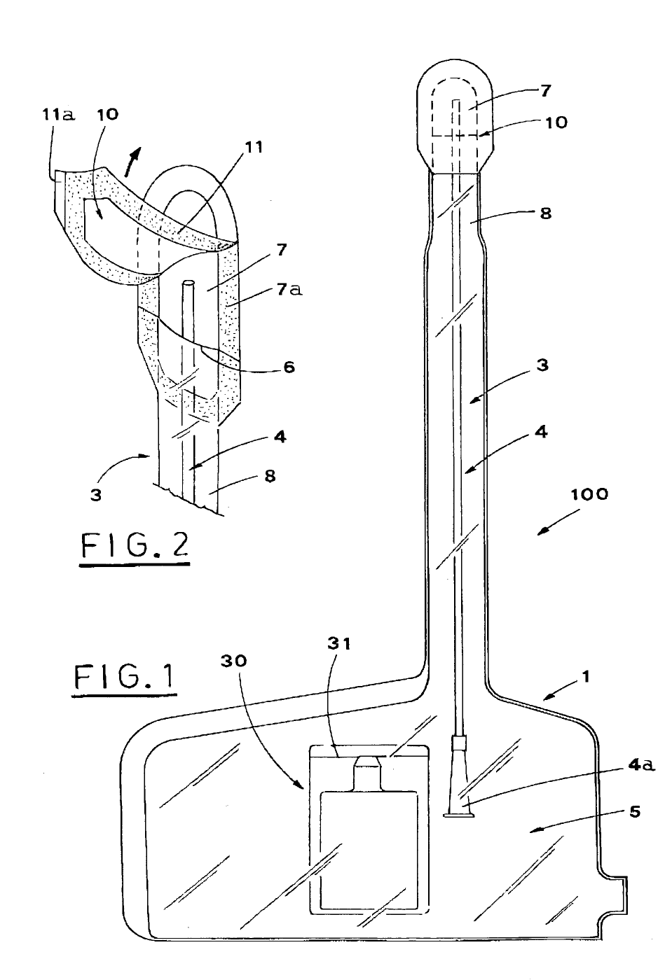

### DESCRIPTION OF THE PREFERRED EMBODIMENT

With reference to the above-mentioned Figures, reference numeral 100 generally indicates a sterilizable device for draining urine, which has been manufactured in accordance with the basic features of the present invention.

The device 100, in particular, includes a bag 1 obtained from two pieces or flaps of flexible plastic material, of the type commonly used for this kind of products, which are superimposed to each other and sealed. In turn, the bag includes a collecting portion 5, for collecting urine coming from the catheter when it is used, and an elongated neck-like portion 3. The elongated portion 3 has a distal end and a proximal end joined to the collecting portion 5.

The bag 1, and in particular the elongated portion 3, is designed to removably retain a catheter 4 therein.

Furthermore, a container 30 is situated inside the collecting portion 5 and is filled with a liquid aimed at activating a lubricating substance applied to the surface of the catheter. A precut line 31 is made in the container for obtaining an easier breakage of the container 30, to allow the liquid contained therein to go out.

The elongated portion 3 has, at its distal end, an opening 6. Furthermore, the distal end of the elongated portion 3 has an extension 7 originated by one of the flap making up the elongated portion, and extending in the longitudinal direction of that single flap of the elongated portion 3, beyond the above-mentioned opening 6.

There is also a border 7a made on the distal end of the elongated portion 3 and along the edge of the extension 7.

The device 100 also includes a segment 10 made from a liquid-tight material. The liquid-tightness condition should be maintained at least for a time period sufficient for the lubricating substance of the catheter 4 to become active. While this material must prevent passage of liquid, however, it can be permeated by gases, which are commonly used for sterilization of the package 100, for instance ethylene oxide. The segment 10 is removably fastened, with its own edge 11 and preferably by means of high-frequency welding, to the distal end of the elongated portion 3 and to the extension 7, along the border 7a, so that the opening 4 is sealed.

A grip portion 11a of the segment 10, outer with reference to the edge 11, is not attached to the elongated portion 3 so as to allow the user to grip the segment and easily remove it.

The segment 10, in particular, is advantageously obtained from a special type of micro-porous paper material which is permeable to gases, which are commonly used for sterilization in medical and para-medical field, of instance ethylene oxide. The material must remain permeable at least for a pre-selected time period. This kind of material is available on the marketplace and is indicated by various brands, which are well known by those skilled in the art.

The elongated portion 3 has, near the opening 6, a narrow section 8, which is capable of engaging and retaining a flared portion 4a of the catheter 4 during its use. In this way, the catheter is prevented from getting out through the opening 6.

Preparation and subsequent use of the draining device 100 as described above, are well evident and easy to conceive, and are described in the following on a general basis only.

While the catheter 4 is being packed inside the bag 1, the segment 10 is applied and fastened to the bag 1 itself, in its proper operative position prior to sterilization of the bag contents. The sterilization gas, usually ethylene oxide, is then blown inside the bag 1. The gas passes through the above-mentioned segment 10, which is permeable to the above-mentioned sterilization gas.

When the catheter 4 is used, the user or anybody who is performing the operation, must first break the container 30 along the precut line 31. Then, once the lubricating substance of the catheter 4 has become active, the segment 4 is removed by a simple operation including gripping the grip portion 11a and tearing the segment away, in the direction as indicated by the arrow in FIG. 2.

The catheter 4 is then extracted up to the point in which the flared portion 4a is retained by the narrow section 8 of the elongated portion 3.

Lastly, usage of the catheter 4 and subsequent disposal of the draining device 100 are preformed in accordance with the usual ways.

The advantage deriving from the present invention basically include an easier opening operation of the draining device 100 and higher safety of the same operation, even when it is performed by unskilled people.

In this way a decrease of the production cost is obtained as well as, consequently, of the sale price.

What is claimed is:

1. A sterilizable draining device for draining urine, including:
a collecting bag, including a urine collecting portion and an elongated neck-like portion, said elengated portion having a distal end and a proximal end joined to said elongated portion;
an opening made at said the distal end of the elongated portion;
a catheter contained within said collecting bag and situated, at least partially, within said elongated portion, such that said catheter can be extracted through said opening when the draining device is used;
at least a segment of liquid-impermeable material, removably fastened to the distal end of said elongated portion such as to seal the opening made therein.

2. A device according to claim 1, wherein said segment is made from micro-porous paper capable of allowing passage of sterilizing gas into said bag.

3. A device according to claim 2, wherein said bag includes two superimposed and sealed pieces or flaps of flexible plastic material and said elongated portion, at a distal end, has an extension projecting from one of said pieces or flaps of said of flexible plastic material beyond said opening, the extension being provided with an enlarged border designed to match with a corresponding edge of said segment.

4. A device according to claim 1, wherein said elongated portion has, near said opening, a narrow section for engaging and retaining a flared portion of said catheter.

5. A device according to claim 1, wherein said segment is fastened to said elongated portion by means of high-frequency welding.
